(19) United States
(12) Patent Application Publication
(10) Pub. No.: US 2005/0047974 A1
(43) Pub. Date: Mar. 3, 2005

(54) Plenum systems

(76) Inventor: Lendell Martin SR., Swansbury Cyress, TX (US)

Correspondence Address: Guy McClung, PMB 347, 16690 Champion Forest Drive, Spring, TX, 77379-7023, US

(21) Appl. No.: 10/652,636

(22) Filed: Aug. 31, 2003

(51) Int. Cl. A61L 9/00

(52) U.S. Cl. 422/121; 422/120; 422/125; 422/4; 422/24

(57) ABSTRACT

An air flow structure for enclosing a coil of an air treatment system, the air flow structure in certain aspects having a container with at least two openings for air flow therethrough, the container having an interior surface, and the interior surface having ultraviolet-resistant material thereon; and methods for using such an air flow structure.

### BACKGROUND OF THE INVENTION

1. Field of the Invention

This invention is directed to air treatment systems; plenums useful in such systems; to enclosures for coils for such systems; and methods for using such systems.

2. Description of Related Art

In many prior art air treatment systems, air is blown by a blower into an enclosure that houses a coil (for heating or for cooling). In a variety of prior art systems an evaporator coil is mounted above a drain pan and a cover covers the top of the coil. A back plate closes off an open side the coil. A fan pulls air from outside the coil and through the coil. In certain aspects there is heat exchange fluid or refrigerant moving within the coil

In many prior art air treatment and air conditioning systems, fiberboard and fiberglass are used as structural materials and as insulating materials. Often raw material, e.g., insulation, fiberglass or fiberboard, exposed in coil enclosures and in air ducts, can be loosened and flow into an air stream into rooms or living areas. The human ingestion of these materials is not desirable.

Many air flow systems now employ ultraviolet lights (e.g. ultraviolet C) to reduce or eliminate mold, bacteria, etc. This light can degrade binder material that binds together insulation pieces and particles and this can result in the loosening of these pieces and particles. Once loosened, these pieces and particles can flow in the air stream and can, eventually, be ingested by animals and humans.

U.S. Pat. Nos. 5,967,228; 5,664,431; 5,284,027; 5,207,074; 5,199,276; 6,276,443; 5,927,096; and 6,311,735 and the references cited in each of these patents (all said patents and references incorporated fully herein for all purposes) disclose various prior art air treatment systems.

There has long been a need for effective and efficient air treatment systems. There has long been a need for effective and efficient coil enclosures and plenums for such systems. There has long been a need for such systems in which insulating material is prevented from flowing into an air stream and into living areas.

### SUMMARY OF THE PRESENT INVENTION

The present invention, in certain embodiments, discloses an air treatment system (for cooling or heating) that has a blower that blows air into a plenum enclosure that houses a heat exchange coil. Any number of openings may be provided through walls of the plenum enclosure for ducting air from the enclosure. In certain particular aspects of the present invention, walls of the enclosure are made of insulating material, or have a layer of insulating material thereon (e.g., fiberglass or fiberboard) with a protective coating, laminate or layer thereon that inhibits or prevents the degradation of material that binds together pieces or particles of the insulating material so that they are dislodged to enter the air exiting from the enclosure.

In certain particular aspects of systems according to the present invention, various parts of the walls, top, and floor of the enclosure have interfitting, interlocking parts for ease of assembly; for added strength; for sealing the enclosure; and rigidity; and for ease of access to the interior of the enclosure.

In certain aspects of the present invention a material is used that reflects ultraviolet light, preventing it from striking material within the enclosure (or within an air duct) thereby reducing degradation of the insulating material and reducing the amount of such material that enters the air flowing from the enclosure (or duct).

In certain particular embodiments, a base pan is included as part of an enclosure according to the present invention. The base pan, in certain aspects has ribs or cross members that increase the stability of the enclosure. Such a base pan also reduces the need for additional bracing or stiffeners.

In certain particular aspects, an enclosure according to the present invention has interfitting, interlocking parts that reduce the need for additional seals or sealing material. Also, in certain aspects, an enclosure according to the present invention has an easily removable top which requires a minimum number of fasteners and which, when in place, seals the top of the enclosure.

In certain embodiments a top side wall, and/or panels (made e.g. metal, fiberboard, and/or fiberglass and/or with a layer of insulating material) are easily removable and, when removed, permit easy installation and removal of a coil within the enclosure. In certain aspects a separate panel or piece of the enclosure has openings through which pass and by which are held in a stable position conduits through which refrigerant (or heat exchange fluid) flows to and from a coil within the enclosure. A removable top on the enclosure can be removed without disturbing the conduits and without disconnecting the conduits.

Adjustable, removable and/or reversible connectors are, optionally, provided for connecting an enclosure according to the present invention to a furnace or other air handling system. In certain aspects this is done without additional fabricated parts, i.e., two pieces interconnect with each other.

In certain aspects systems according to the present invention have an accessory auxiliary drain pan in addition to a main drain pan.

In certain embodiments, the present invention discloses a coil for use in air treating apparatuses. The coil includes two expansion valves, each with a corresponding gas temperature bulb sensing device. Each expansion valve is associated with one side or slab of a two-sides or “V-shaped” coil. By providing two expansion valves differences in air flow through one side as opposed to the other can be addressed so that, as needed, more cooling fluid is provided to a side through which there is more air flow. In other aspects, one expansion valve system controls refrigerant flow to the front ends of both sides of the coil (or tops depending on coil orientation) and the other controls such flow to the rear ends (or bottoms) of the coil sides.

What follows are some of, but not all, the objects of this invention. Objects other than the specific objects stated below, additional objects and purposes will be readily apparent to one of skill in this art who has the benefit of this invention's teachings and disclosures. It is, therefore, an object of at least certain preferred embodiments of the present invention to provide new, useful, unique, efficient and nonobvious air treatment systems and new, useful, unique, efficient and nonobvious coils and coil enclosures; plenums for them; and systems that use them;

Such systems with stabilized insulating material which is inhibited from entering air exiting from a duct or an enclosure or plenum;

Such systems in which deleterious effects of ultraviolet light on binder material binding together insulation and/or structural material are reduced;

Such systems with material that inhibits the absorption of ultraviolet light to reduce the degradation of the material and inhibit the loosening and freeing of pieces and particles of the material;

Such a system with an enclosure or plenum with interlocking parts that ease assembly, increase strength, seal an enclosure, and/or facilitate access to the enclosure's interior; and

Coils with multiple sides, each side with an expansion valve system to compensate for different air flow volummes through different coil sides; and

New, useful, unique, efficient and nonobvious methods of using such plenums, enclosures, and systems.

Certain embodiments of this invention are not limited to any particular individual feature disclosed here, but include combinations of them distinguished from the prior art in their structures and functions. Features of the invention have been broadly described so that the detailed descriptions that follow may be better understood, and in order that the contributions of this invention to the arts may be better appreciated. There are, of course, additional aspects of the invention described below and which may be included in the subject matter of the claims to this invention. Those skilled in the art who have the benefit of this invention, its teachings, and suggestions will appreciate that the conceptions of this disclosure may be used as a creative basis for designing other structures, methods and systems for carrying out and practicing the present invention. The claims of this invention are to be read to include any legally equivalent devices or methods which do not depart from the spirit and scope of the present invention.

The present invention recognizes and addresses the previously-mentioned problems and long-felt needs and provides a solution to those problems and a satisfactory meeting of those needs in its various possible embodiments and equivalents thereof. To one of skill in this art who has the benefits of this invention's realizations, teachings, disclosures, and suggestions, other purposes and advantages will be appreciated from the following description of preferred embodiments, given for the purpose of disclosure, when taken in conjunction with the accompanying drawings. The detail in these descriptions is not intended to thwart this patent's object to claim this invention no matter how others may later disguise it by variations in form or additions of further improvements.

### DESCRIPTION OF THE DRAWINGS

A more particular description of embodiments of the invention briefly summarized above may be had by references to the embodiments which are shown in the drawings which form a part of this specification. These drawings illustrate certain preferred embodiments and are not to be used to improperly limit the scope of the invention which may have other equally effective or legally equivalent embodiments.

FIG. 1A is a perspective view of an air treatment enclosure according to the present invention. FIG. 1B is an exploded view of the enclosure of FIG. 1A (with side walls deleted).

FIG. 2A is a top view, FIG. 2B is a side view, FIG. 2C is a bottom view, FIG. 2D is a bottom view, and FIGS. 2E and 2F are end views of the enclosure of FIG. 1A.

FIG. 3 is a perspective view of the enclosure of FIG. 1A.

FIG. 4 is a partial perspective, partial cutaway view of parts of the enclosure of FIG. 1A.

FIG. 5 is a partial perspective view of parts of the enclosure of FIG. 1A.

FIG. 6 is a partial cross-sectional view of the enclosure of FIG. 1A.

FIG. 7 is a cross-sectional view of insulating material according to the present invention.

FIG. 8A is a perspective view of a coil according to the present invention. FIG. 8B is a front view and FIG. 8C is a rear view of the coil of FIG. 8A. FIG. 8D is a top view of the coil of FIG. 8A.

### DESCRIPTION OF EMBODIMENTS PREFERRED AT THE TIME OF FILING FOR THIS PATENT

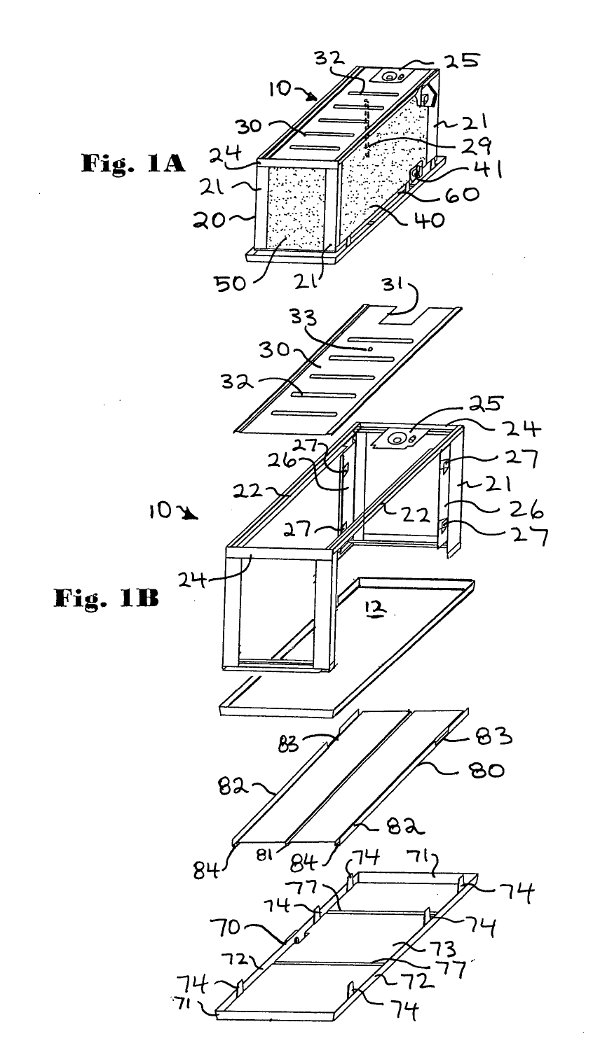

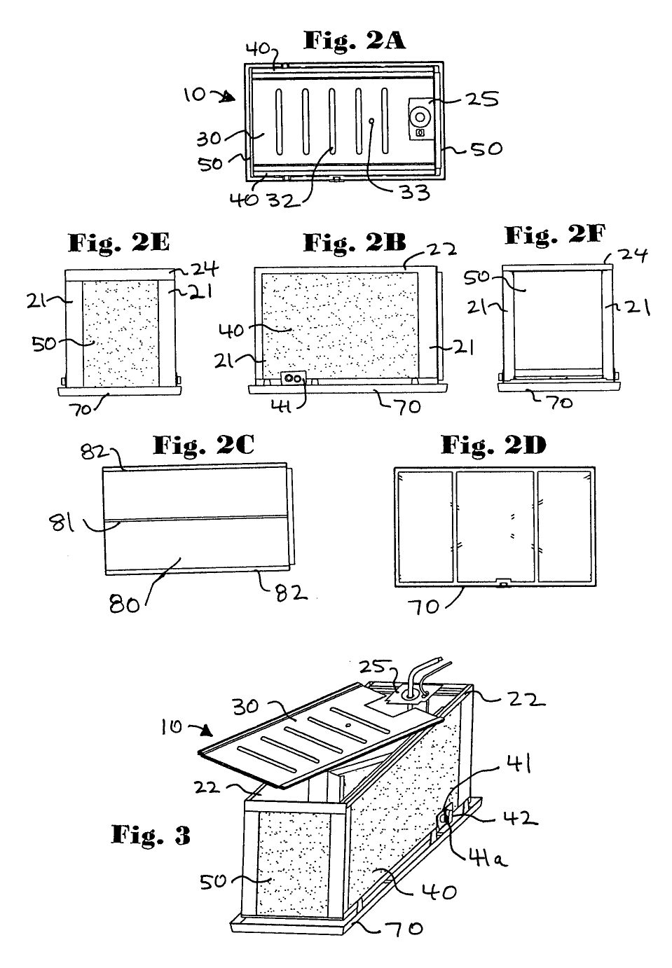

FIGS. 1A-2D show an enclosure 10 according to the present invention which has a central structure 20, a top 30, side panels 40, end panels 50, base 60, an auxiliary drain pan 70, and a primary drain pan 12 (which may be any suitable known drain pan, including, but not limited to, those from Allstyle Coil Co., Houston, Tex., and those disclosed in U.S. Pat. No. 5,987,909 and in the prior art cited therein. The central structure 20 has interconnected vertical members 21, top rails 22, bottom members 23, and top members 24. The top 30 is removable and releasably interlocks with the top rails 22 and has a cut-out portion 31 which slides under a conduit panel 25 which is attached to one of the top members 24. The mounts 26 connected to vertical members 21 have bent members 27 for holding a coil.

The top 30 has a series of spaced-apart ribs 32 (made manually in a piece of material or formed integrally thereof in a manufacturing process, e.g. an extrusion process) which render the top 30 more rigid. An opening 33 provides an opening for insertion of an ultraviolet lamp 29 or for passage therethrough of an electrical wire or cable for such a lamp.

An optional base 80 has a central rib 81 formed by making recess in the base 80 and sides 82 which strengthen and rigidify the base 80. Cut-out portions 83 in the sides 82 accommodate drain apparatus 41. Recesses 84 in the sides 82 receive and hold part of the lower ends of the vertical members 21 of the central structure 20.

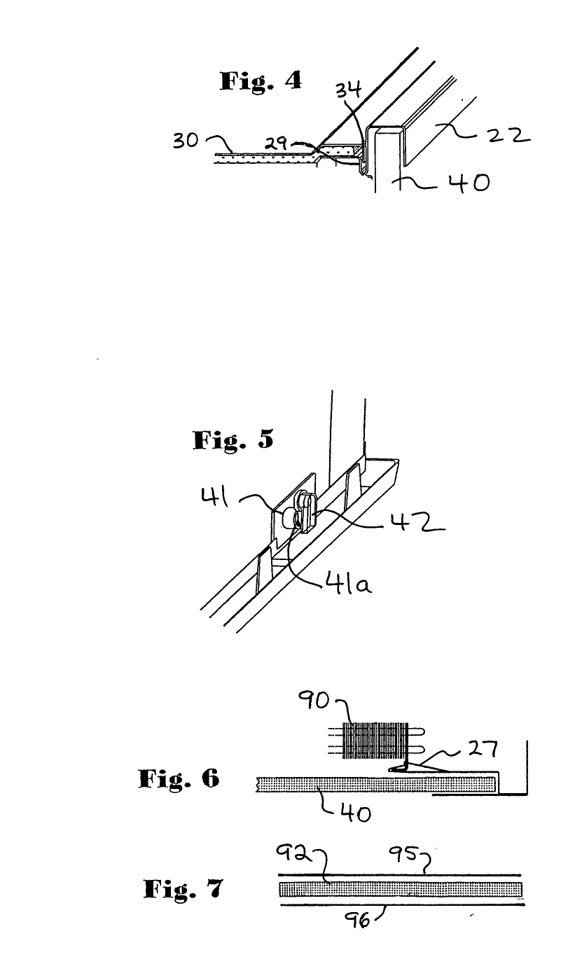

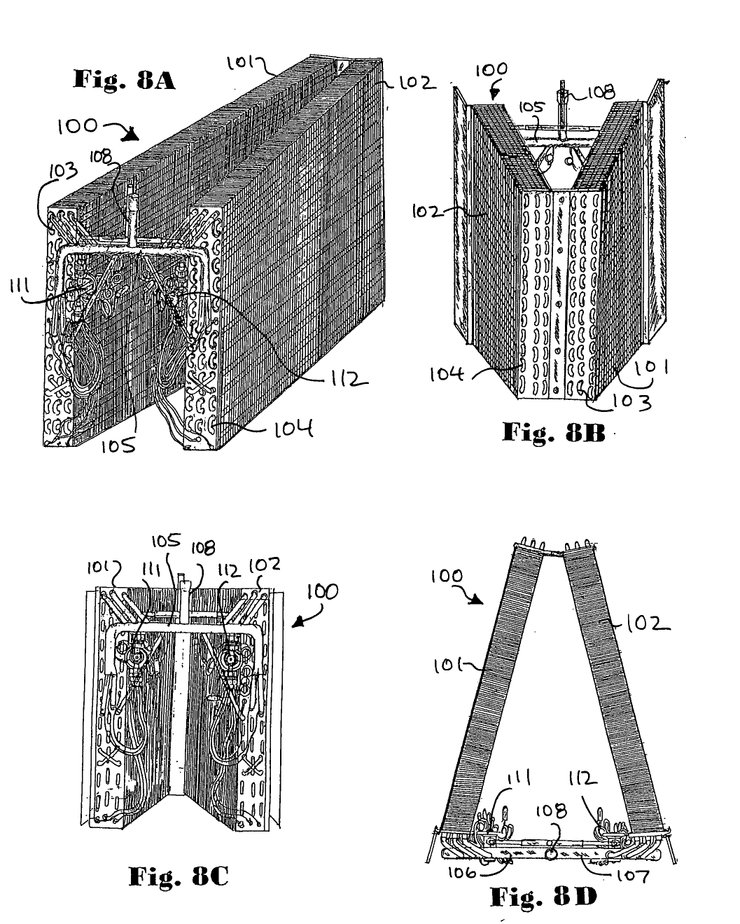

The auxiliary drain pan 70 has two spaced-apart upright end members 71; two spaced-apart upright sides 72; a bottom 73; and two spaced-apart ribs 77. A plurality of spaced-apart vertical members 74 connected to or formed integrally of the sides 72 help to maintain the enclosure in position on the auxiliary drain pan 70. in certain aspects the upright members 74 are used to support the enclosure so that it does not directly rest on or contact the bottom of the auxiliary pan. Screws extending through the members 74 extend into the base of the enclosure. This also provides an airway between the enclosure's bottom (or the primary pan) and the auxiliary pan 70 so that air can flow between them elevating the dew point, reducing condensation, and thereby reducing or inhibiting the growth of mold, bacteria, etc. As shown in FIGS. 1A and 3, the central structure 20 sits on base 80 (and/or primary pan) and they both are mounted on the drain pan 70 and the central structure 20 is encompassed within the vertical members 74.

Retainer clips 27 releasably hold a coil in place within the enclosure and insure its proper positioning.

As shown in FIGS. 1A and 5, a primary drain 41 extends over a cut-out portion 83 of the base 80. The drain 41 has a drain opening 41a that can, selectively, be plugged off with a plug 42 that is positioned to the side of the opening 41a on a shaft (not shown) for easy removal from the shaft and installation plugging off the opening 41a.

FIG. 4 shows the fit between the top 30 and the rails 22. Lips 34 of the top 30 are received and held in a recess 29 of the rails 22. It is within the scope of this invention for the top 30 to have attached or adhered thereto a layer of insulation material 35, e.g., but not limited to, a layer of fiberglass about one-half an inch thick which is glued to the top 30.

FIG. 6 shows a heat exchange coil 90 within an enclosure like the enclosure 10. The coil 90 is held releasably in place with retainer clips, e.g. with clips 27 (e.g. as do the clips 27 in FIG. 1B).

FIG. 7 shows a structural member 92 according to the present invention (exploded view) which has a coating 95 of “FSK” (commercially available “foil-skim-Kraft” material) and a coating 96 of ultraviolet-light-resistant material, e.g. ultraviolet-resistant laminate, plastic, or foil (e.g. metal or polyester) (each coating about one to four mils). Instead of the FSK material, MYLAR™ material or a suitable vapor barrier material may be used. The FSK and/or ultraviolet-resistant material may be applied with suitable adhesives and know application methods. It is within the scope of the present invention for any panel of any enclosure according to the present invention and for any layer of insulation to have a coating 95 and/or a coating 96 (or to have both coatings on both the exterior and the interior of the layer. It is also within the scope of the present invention for any air duct in any air conditioning system to have any such layer and/or for exposed insulation material or structural material therein to have one or two such coatings on its interior and/or exterior. It is within the scope of this invention for the insulation to be fiberglass, foam, mineral wool, or cellulose.

The various parts of enclosures according to the present invention may be made of fiberglass, composite, plastic, steel, iron, aluminum, aluminum alloys, galvanized material, zinc, and/or zinc alloys. The various pans and panels may be made of plastic, e.g. polypropylene or polycarbonate. Preferably, an assembled enclosure has extremely little or no fiberglass or fiberboard material which directly receives ultraviolet light. Glues are employed, in certain aspects, (e.g. to glue insulation and/or ultraviolet-resistant material to structural pieces) which are moisture resistant and/or fire retardant. One useful polyester foil used with components of the present invention is polyester foil laminate provided by Cleveland Laminating Corp. In one particular aspect panels in systems according to the present invention have an exterior MYLAR™ coating and an interior polyester foil lamination.

FIGS. 8A-8E show a coil system 100 for an air treatment system which has two sides 101, 102 or slabs (e.g. as disclosed in U.S. Pat. Nos. 5,987,909; 6,519,966; 6,276,443; 5,284,027; and 5,664,431 all incorporated fully herein for all purposes). Heat exchange fluid flows through multiple tubes 103, 104 respectively in each side 101, 102. Depending on the coil orientation (e.g. flat as shown in FIG. 8A or on end with the narrower V-tip at the top) different portions or parts of the coil can have different amounts of air flowing therethrough. The system 100 has two expansion valve systems 111, 112 to address this situation and provide an adequate amount of heat exchange fluid to each part or portion of the system 100.

Heat exchange fluid is provided to the system 100 via a line 108 that flows into a manifold 105 with one branch 106 in fluid communication with the expansion valve system 111 and another branch 107 in fluid communication with the expansion valve system 112. Each expansion valve system controls heat exchange fluid flow to specific tubes which, in turn, traverse specific parts or portions of the system 100. For example, as shown in FIG. 8B with the system 100 in a generally horizontal orientation with air flowing into the open end of the “V” as viewed in FIG. 8B and then out of the two sides 101, 102, the expansion valve system 100 controls fluid flow to tubes in approximately the back half (or three-fourths) of the system 100 and the expansion valve system 102 controls fluid flow to tubes in the approximately front half (or one-fourth) of the system 100. Such an arrangement can be beneficial when, as is often the case, there is a higher air pressure within the front of the system 100 and, therefore, higher air flow through this part with a corresponding higher demand for circulated heat exchange fluid.

The present invention, therefore, in some, but in not necessarily all embodiments, provides an air treatment system with air flow structure for enclosing a coil of an air treatment system, the air flow structure, in at least certain aspects, having a container with at least two openings for air flow therethrough, the container having an interior surface, the interior surface having ultraviolet-resistant material thereon.

In conclusion, therefore, it is seen that the present invention and the embodiments disclosed herein and those covered by the appended claims are well adapted to carry out the objectives and obtain the ends set forth. Certain changes can be made in the subject matter without departing from the spirit and the scope of this invention. It is realized that changes are possible within the scope of this invention and it is further intended that each element or step recited in any of the following claims is to be understood as referring to all equivalent elements or steps. The following claims are intended to cover the invention as broadly as legally possible in whatever form it may be utilized. The invention claimed herein is new and novel in accordance with 35 U.S.C. § 102 and satisfies the conditions for patentability in § 102. The invention claimed herein is not obvious in accordance with 35 U.S.C. § 103 and satisfies the conditions for patentability in § 103. This specification and the claims that follow are in accordance with all of the requirements of 35 U.S.C. § 112. The invention claimed herein is not obvious in accordance with 35 U.S.C. § 103 and satisfies the conditions for patentability in § 103. This specification and the claims that follow are in accordance with all of the requirements of 35 U.S.C. § 112. The inventor may rely on the Doctrine of Equivalents to determine and assess the scope of the invention and of the claims that follow as they may pertain to apparatus not materially departing from, but outside of, the literal scope of the invention as set forth in the following claims.

1. An air flow structure for enclosing a coil of an air treatment system, the air flow structure comprising
a container with at least two openings for air flow therethrough,
the container having an interior surface,
the interior surface having ultraviolet-resistant material thereon.

2. The air flow structure of claim 1 wherein the container includes structural members made of material bound together with binder material, said binder material degradable by ultraviolet light.

3. The air flow structure of claim 1 wherein the container is a coil enclosure.

4. The air flow structure of claim 1 wherein the container is a duct for transmitting air.

5. The air flow structure of claim 1 further comprising
coil apparatus within the container for exchanging heat between the coil apparatus and air flowing through the coil apparatus.

6. The air flow structure of claim 5 wherein the coil apparatus has at least a first part and a second part, the air flow structure further comprising
first expansion valve apparatus for selectively controlling flow of heat exchange fluid through the first part of the coil apparatus, and
second expansion valve apparatus for selectively controlling flow of heat exchange fluid through a second part of the coil apparatus.

7. The air flow structure of claim 1 further comprising
a light source within the container for providing ultraviolet light to air flowing therethrough.

8. The air flow structure of claim 1 wherein the ultraviolet resistant material comprises laminate material.

9. The air flow structure of claim 1 wherein the container is made of material from the group consisting of metal, plastic, wood, fiberglass, and fiberboard.

10. The air flow structure of claim 1 wherein the container has an interior surface and substantially all of the interior surface is protected by the ultraviolet-resistant material.

11. The air flow structure of claim 1 wherein the container is made of structural material with the interior surface and with an outer surface, the outer surface having a layer of vapor barrier material thereon.

12. An air flow structure for enclosing a coil of an air treatment system, the air flow structure comprising
a container with at least two openings for air flow therethrough,
the container having an interior surface,
the interior surface having ultraviolet-resistant material thereon,
wherein the container includes structural members made of material bound together with binder material, said binder material degradable by ultraviolet light,
wherein the container is a coil enclosure,
coil apparatus within the container for exchanging heat between the coil apparatus and air flowing through the coil apparatus, and
a light source within the container for providing ultraviolet light to air flowing therethrough.

13. The air flow structure of claim 12 further comprising
coil apparatus within the container for exchanging heat between the coil apparatus and air flowing through the coil apparatus.

14. The air flow structure of claim 12 further comprising
first expansion valve apparatus for selectively controlling flow of heat exchange fluid through the first part of the coil apparatus, and
second expansion valve apparatus for selectively controlling flow of heat exchange fluid through a second part of the coil apparatus.

15. A method for treating air, the method comprising
flowing air through an air flow structure, the air flow structure comprising a container with at least two openings for air flow therethrough, the container having an interior surface, and the interior surface having ultraviolet-resistant material thereon.

16. The method of claim 15 wherein the air flow structure includes coil apparatus within the container for exchanging heat between the coil apparatus and air flowing through the coil apparatus, first expansion valve apparatus for selectively controlling flow of heat exchange fluid through the first part of the coil apparatus, and second expansion valve apparatus for selectively controlling flow of heat exchange fluid through a second part of the coil apparatus, the method further comprising
selectively controlling the flow of heat exchange fluid through the coil apparatus with the first expansion valve apparatus and the second expansion valve apparatus.

17. The method of claim 15 wherein the air flow structure includes coil apparatus within the container for exchanging heat between the coil apparatus and air flowing through the coil apparatus, first expansion valve apparatus for selectively controlling flow of heat exchange fluid through the first part of the coil apparatus, and second expansion valve apparatus for selectively controlling flow of heat exchange fluid through a second part of the coil apparatus, the method further comprising
selectively controlling the flow of heat exchange fluid through the coil apparatus with the first expansion valve apparatus and the second expansion valve apparatus.

18. Coil apparatus for an air treatment system, the coil apparatus comprising
the coil apparatus positionable within a container for exchanging heat between the coil apparatus and air flowing through the coil apparatus,
the coil apparatus having at least a first part and a second part,
first expansion valve apparatus for selectively controlling flow of heat exchange fluid through the first part of the coil apparatus, and
second expansion valve apparatus for selectively controlling flow of heat exchange fluid through a second part of the coil apparatus.
